(19) United States
(12) Patent Application Publication
(10) Pub. No.: US 2021/0308432 A1
(43) Pub. Date: Oct. 7, 2021

(54) LENGTH-ADJUSTABLE CATHETER AND METHOD THAT EMPLOYS A LENGTH-ADJUSTABLE CATHETER TO TREAT VASCULAR PATHOLOGIES

(71) Applicant: CTI Vascular AG, Neuhausen (CH)

(72) Inventors: Marc Gianotti, Wiesendangen (CH); Michael Jetter, Thayngen (CH); Martin Rickert, Rottwell (DE); Dragana Gajic, Schaffhausen (CH); Sabina Silva, Diessenhofen (CH); Andreas Bodmer, Windisch (CH); Valentin Nickel, Singen-Schlatt (DE); Ulf Fritz, Tengen (DE)

(73) Assignee: CTI Vascular AG, Neuhausen (CH)

(21) Appl. No.: 17/236,916

(22) Filed: Apr. 21, 2021

Related U.S. Application Data
(63) Continuation of application No. 16/752,999, filed on Jan. 27, 2020.
Application No. 16/752,999 is a continuation-in-part of application No. 15/502,729, filed on Feb. 8, 2017, now Pat. No. 10,688,276.

(51) Int. Cl. A61M 25/10

(52) U.S. Cl. CPC A61M 25/104; A61M 2025/1068; A61M 2205/582; A61M 2205/583; A61M 2205/32; A61M 2205/0216; A61M 2025/1056

(57) ABSTRACT

The current document is directed to length-adjustable catheters and methods that employ length-adjustable catheters to treat malformations, constrictions, obstructions, lesions, and blockages within patients' blood vessels. The length of the shaft of a length-adjustable catheter, to which the current application is directed, can be adjusted over a range of lengths prior to and during medical procedures. In many implementations, length adjustment is accompanied by indications, to the medical provider, of the extent of a length adjustment. The indications may include one or more of visual markings, haptic feedback, radio-opaque markings, and/or other types of indications. In many implementations, the variable-length mechanism of the length-adjustable catheter is mechanically lockable following length adjustment.

### CROSS-REFERENCE TO RELATED APPLICATIONS

This application is a continuation of application Ser. No. 16/752,999, filed Jan. 27, 2020, which is a continuation-in-part of application Ser. No. 15/502,729, filed Feb. 8, 2017, now U.S. Pat. No. 10,688,276, issued Jun. 23, 2020, which claims priority to PCT/IB2015/001981, filed Aug. 26, 2015.

### TECHNICAL FIELD

The current document is directed to catheters used to treat vascular pathologies and, in particular, to catheters with variable shaft lengths that are used for treating a variety of different vascular conditions.

### BACKGROUND

Angioplasty balloon catheters have been developed to treat a variety of different manifestations of vascular disease within patients' veins and arteries that, when not treated, often lead to increasingly serious health conditions and complications, including ischemia, heart attacks, embolisms, and strokes. An angioplasty balloon catheter is generally inserted, along a previously inserted guide wire, into a patient's blood vessel at a variety of different blood-vessel access points, including the femoral, subclavian, radial, and brachial arteries. The catheter is advanced along the guide wire in order to position the inflatable portion of the angioplasty balloon catheter in or near a target region of the blood vessel. The balloon is then inflated in order to mechanically dilate and displace a blockage, lesion, or other problem within the target region. Currently available angioplasty balloon catheters have fixed shaft lengths.

As a result, a treatment provider generally selects a balloon angioplasty catheter of appropriate length in order to access a particular treatment site from a particular access point. However, in many procedures, a vessel is blocked at more than one point. In many cases, the treatment provider therefore needs to employ two or more angioplasty balloon catheters of two or more different lengths in order to reach and ameliorate the two or more blockages from the particular access point. Because angioplasty-balloon-catheter-based procedures involve prior insertion of the guide wire, insertion and removal of multiple, different-length angioplasty balloon catheters may result in a variety of cascading complexities and problems, including a need to remove and reinsert different-length guide wires, complexities associated with maintaining sterile protocols across multiple sub-procedures, increased procedure times, and potential for additional complications arising from additional procedural steps, including risk of vessel damage and unintentional vessel-wall penetration. Designers, developers, and treatment providers therefore continue to seek new and improved treatment equipment and associated methods for treating blood-vessel malformations, constrictions, obstructions, lesions, and blockages.

### SUMMARY

The current document is directed to length-adjustable catheters and methods that employ length-adjustable catheters to treat malformations, constrictions, obstructions, lesions, and blockages within patients' blood vessels. The length of the shaft of a length-adjustable catheter, to which the current application is directed, can be adjusted over a range of lengths prior to and during medical procedures. In many implementations, length adjustment is accompanied by relative-position indications, to the medical provider, of the extent of a length adjustment. The indications may include one or more of visual markings, haptic feedback, radio-opaque markings, and/or other types of indications. In many implementations, the variable-length mechanism of the length-adjustable catheter is mechanically lockable following length adjustment.

### BRIEF DESCRIPTION OF THE DRAWINGS

FIGS. 1A-B illustrate a first implementation of a length-adjustable angioplasty balloon catheter.

FIGS. 2A-B illustrate a second implementation of the length-adjustable angioplasty balloon catheter.

FIG. 3 shows a third implementation of the length-adjustable angioplasty balloon catheter.

FIG. 4 illustrates a fourth implementation of the length-adjustable angioplasty balloon catheter. FIGS. 5A-B illustrate a fifth implementation of the length-adjustable angioplasty balloon catheter.

FIGS. 6-11 show a variety of different implementations of dual-lumen and single-lumen shaft-tube engagement configurations and locking mechanisms.

FIG. 12 illustrates an engagement configuration and locking mechanism that, as one example, may be employed in the length-adjustable angioplasty balloon catheter implementation shown in FIGS. 5A-B.

FIG. 13 shows, in cross-section, a fitting that provides a fluid-impermeable slidable seal to facilitate sliding of a smaller-diameter portion of a length-adjustable catheter shaft within a larger-diameter portion of the catheter shaft.

FIG. 14 shows an additional engagement configuration and locking mechanism.

FIG. 15 shows a series of steps carried out by a treatment provider, using a length-adjustable angioplasty balloon catheter, to treat multiple blockages or lesions within a patient's blood vessel.

FIG. 16 illustrates insertion of an introducer sheath into a patient's blood vessel followed by insertion of a guide wire.

FIG. 17 illustrates advancement of a guide wire into a patient's blood vessel across a target treatment site.

FIG. 18 illustrates introduction of a length-adjustable catheter over a guide wire through an introducer into a patient's blood vessel.

FIG. 19 illustrates length-adjustable catheter extension.

FIG. 20 illustrates advancement of an extended length-adjustable catheter to a second target area.

FIG. 21 illustrates a sixth implementation of the length-adjustable angioplasty balloon catheter.

FIGS. 22A-C show, in cross-section, two fitting configurations that describe the fluid-impermeable seal introduced in FIG. 21 and an additional three-dimensional representation of the configuration shown in FIG. 22A.

FIG. 23 illustrates a dual-lumen reinforced shaft configuration adaptable to the currently disclosed length-adjustable catheters.

FIG. 24 shows an engagement, locking and position-indicating configuration that, as one example, may be employed in the length-adjustable angioplasty balloon catheter implementation shown in FIG. 21.

FIG. 25 illustrates a position-indicating configuration adaptable to the currently disclosed length-adjustable catheters.

### DETAILED DESCRIPTION

The current document is directed to a variety of different implementations of catheters with length-adjustable shafts that are used to treat vascular pathologies. The length-adjustable catheters facilitate both treatments of single target sites within blood vessels as well as procedures that involve treatment of multiple target sites. Because the shaft length of a length-adjustable catheter can be changed during a medical procedure, after the catheter has been initially inserted into a patient's blood vessel, the length-adjustable catheter provides for adjustment of initial non-optimal placements, changes to which might otherwise involve removal of an initially inserted first catheter and reinsertion of a second catheter with a different length. The catheter significantly simplifies treatments of multiple target sites within a blood vessel, since the length of the length-adjustable catheter can be changed, during a procedure, following treatment of a first target site, in order to reposition the catheter to treat a second target site. Length-adjustable catheters may also reduce needed equipment inventories, since fewer different length-adjustable catheters are needed to span the potential range of usable lengths needed for accessing variably positioned treatment sites encountered in human anatomy. In the following discussion, examples of length-adjustable angioplasty balloon catheter are illustrated and described. Angioplasty balloon catheters are commonly employed in a variety of medical treatments. The length-adjustability discussed with reference to the various implementations of angioplasty balloon catheters can be applied to, and incorporated within, other types of catheters used for diagnostic and therapeutic procedures.

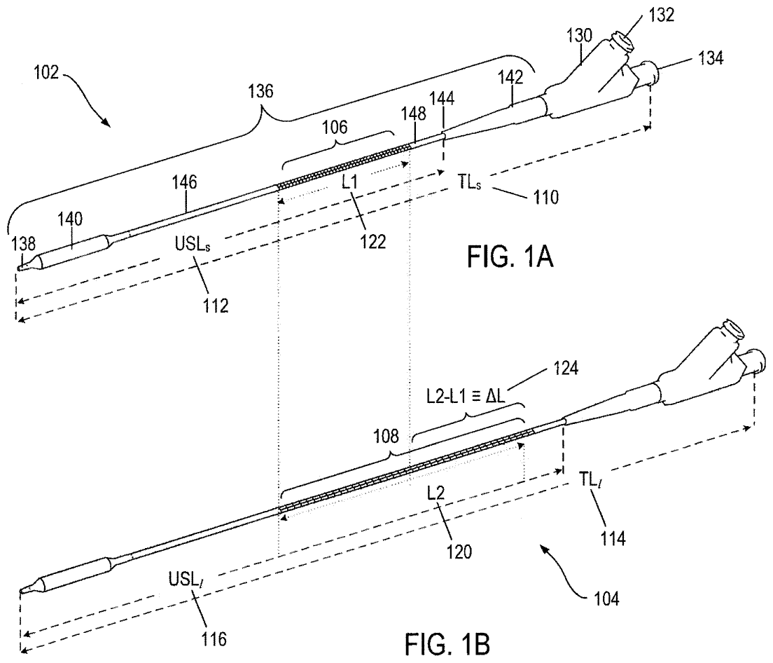

FIGS. 1A-B illustrate a first implementation of a length-adjustable angioplasty balloon catheter. FIG. 1A shows the length-adjustable angioplasty balloon catheter 102 in a fully contracted configuration and FIG. 1B shows the length-adjustable angioplasty balloon catheter 104 in a fully extended configuration. The range of lengths that can be adopted by the length-adjustable angioplasty balloon catheter arises from extension and contraction of a variable-length portion of the length-adjustable-catheter shaft shown as length-adjustable portion 106 in FIG. 1A and length-adjustable portion 108 in FIG. 1B. When fully contracted, as shown in FIG. 1A, the length-adjustable catheter 102 has a total length, TL₅. 110 and a shorter usable length, USL₅ 112. When the length-adjustable portion of the shaft is fully extended, as shown in FIG. 1B, the length-adjustable catheter has a total length, TL₁, 114 and a shorter usable length, USL₁ 116. As is readily apparent by comparing FIG. 1A and FIG. 1B, TL₁ is greater than TL₅ and USL₁ is greater than USL₅. The difference in length between the contracted configuration 102 and the extended configuration 104 can be seen, by comparing FIGS. 1A and 1B, to be equal to the difference between the extended length L2 (120 in FIG. 1B) of the variable-length portion of the shaft and the length L1 (122 in FIG. 1A) of the contracted variable-length portion of the shaft. This difference, L2-L1, is shown in FIG. 1B as a portion 124 of the extended variable-length portion of the shaft 108 not overlapped by the length of the contracted variable-length portion of the shaft 122 when the two adjustable-catheter configurations 102 and 104 are parallel and aligned. Thus, both the usable length and total length of the length-adjustable angioplasty balloon catheter can be adjusted over a variable-length adjustment range of L2-L1. Both the fully contracted length L1 and the fully extended length L2 of the variable-length portion of the length-adjustable-catheter shaft (106 and 108 in FIGS. 1A-B) may differ in different implementations. As is discussed with reference to subsequent figures, the variable-length portion of the length-adjustable catheter may have different sizes, configurations, shapes, and constructions in different implementations.

The various components and features of the length-adjustable angioplasty balloon catheter are next described with reference to FIG. 1A. The length-adjustable catheter includes a manifold 130 with two ports: (1) an inflation port 132; and (2) a guide-wire port 134. The manifold is mounted over a first end of a catheter shaft 136. In the implementation shown in FIG. 1A, the catheter shaft 136 includes two internal hollow bores, or lumens: (1) a first inflation lumen connected to the inflation port 132; and (2) a second guide-wire lumen connected to the guide-wire port 134. The guide-wire lumen extends from the guide-wire port 134 to the second end, or tip, 138 of the catheter shaft. The adjustable-length catheter is slidably mounted onto a guide wire and translated in either direction along the guide wire during insertion of a portion of the shaft into a patient's blood vessel. The inflation lumen is connected to the inflation port 132 and extends along the catheter shaft to an inflatable catheter balloon 140. Liquids and/or gases, including contrast-agent and saline formulations, air, and other such gases and/or liquids, are transferred, under positive pressure, from the inflation port 132 through the inflation lumen to the inflatable catheter balloon 140, resulting in inflation of the catheter balloon. The various liquids and/or gases are transferred, under negative pressure, from the inflated catheter balloon 140 back through the inflation lumen and out through the inflation port 132, deflating the catheter balloon. In the implementation shown in FIG. 1A, the inflation lumen lies above the guide-wire lumen. The inflation lumen and guide-wire lumen are completely separate from one another, so that there is no possibility for leakage of the liquids and/or gases transported by the inflation lumen into the guide-wire lumen or for leakage of fluids within the guide-wire lumen into the inflation lumen.

In the implementation shown in FIG. 1A, a portion of the catheter shaft close to the manifold is covered by a kink-protection sleeve 142. The usable length of the length-adjustable catheter, USL₅, in the configuration shown in FIG. 1A, extends from the tip of the kink-protection sleeve 144 to the catheter tip 138. In the implementation shown in FIG. 1A, the external surfaces of guide-wire and inflation tubes that form internal guide-wire and inflation lumens, respectively, are exposed and visible in the variable-length portion 106, that are covered by an enclosing tube or sheath 146 and 148 on either side of the variable-length portion 106. It should be noted that these tubes may not necessarily be separately manufactured or formed, but may, instead, be portions of a single molded or extruded double-lumen shaft component. In certain implementations, the two enclosing sheaths 146 and 148 have equal outside diameters, while in other implementations, the outside diameters of the two enclosing sheaths 146 and 148 may differ. The variable-length portion (106 and 108 in FIGS. 1A-B) of the catheter shaft is longitudinally and plastically deformable, having a folding pattern embedded into, or imprinted or molded on, the outer surface to facilitate longitudinal extension and contraction. In the implementation shown in FIG. 1A, the inflation tube is parallel to, and positioned above, the guide-wire tube. In alternative implementations, the two tubes may be coaxial, with the guide-wire tube nested within the larger-diameter inflation tube. In certain implementations, the inflation and guide-wire tubes are continuous, comparably rigid tubes, with the portion of the tubes corresponding to the variable-length portions of the shaft patterned or otherwise modified to provide for longitudinal extension and contraction. In alternative implementations, the inflation tube and guide-wire tube each comprises multiple sections of tubing, with middle tube sections corresponding to the variable-length portions of the inflation tube and guide-wire tube each coupled or affixed, at both ends, to tubes corresponding to the non-variable-portions of the inflation tube and guide-wire tube.

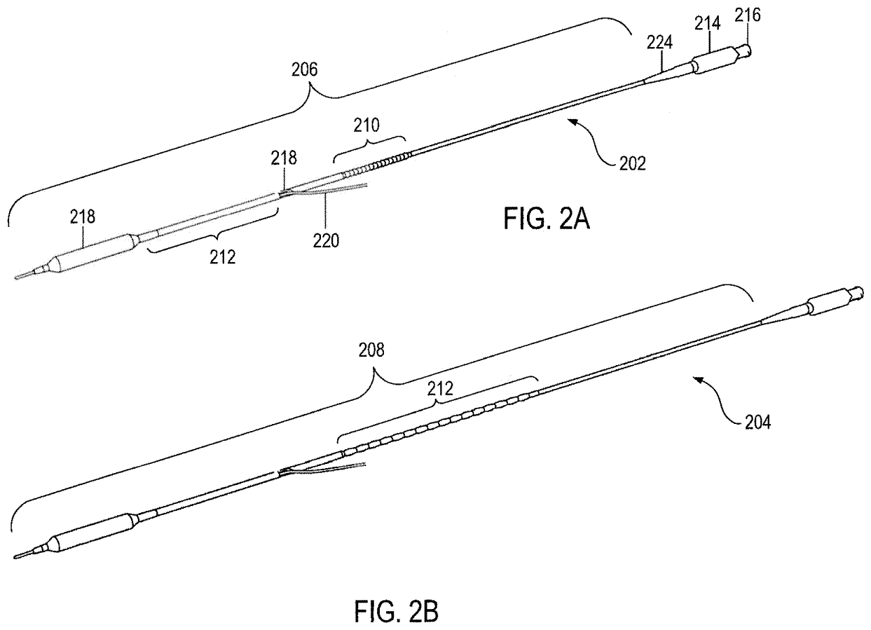

FIGS. 2A-B illustrate a second implementation of the length-adjustable angioplasty balloon catheter. In the implementation shown in FIGS. 2A-B, with a contracted configuration 202 shown in FIG. 2A and an extended configuration 204 shown in FIG. 2B, the catheter shaft 206 and 208 has a variable-length portion 210 and 212, similar to the variable-length portion of the catheter shaft of the implementation shown in FIGS. 1A-B. However. the shaft of the length-adjustable catheter implementation shown in FIGS. 2A-B includes two parallel lumens for only a portion 212 of the catheter shaft, and features a smaller manifold 214 with only a single inflation port 216 rather than the two-port manifold (130, 132, and 134 in FIG. 1A) of the first implementation. This type of implementation is referred to as a “rapid exchange” or “RX” configuration. In the implementation shown in FIGS. 2A-B, a guide-wire port 218 introduces a guide wire 220 into the dual-lumen portion 212 of the catheter shaft. In the implementation shown in FIGS. 2A-B, the outer diameter of the catheter shaft 212 between the variable-length portion 210 and the inflatable catheter balloon 222 has a larger outer diameter than the portion of the catheter shaft between the variable-length region 210 and the kink-protection sleeve 224. In one implementation, the smaller-diameter portion of the catheter shaft comprises a metal hypotube while the larger-diameter portion of the catheter shaft has a polymeric composition. In this implementation, in the dual-lumen portion of the catheter shaft, the inflation tube and guide-wire tube run parallel to one another, and, in various implementations, may have different relative dispositions. In certain implementations, the variable-length portion 210 of the catheter shaft is a separately formed member that is coupled or attached to additional catheter-shaft components, at both ends. In other implementations, the entire guide-wire lumen is a single tube with modifications to the variable-length portion to render the variable-length portion longitudinally extendable and compressible. In other implementations, the portion of the catheter shaft between the inflatable catheter balloon 222 and the variable-length portion 210 may slide over the variable-length portion 210 and, in additional implementations, over additional portions of the catheter shaft, including the single-port manifold.

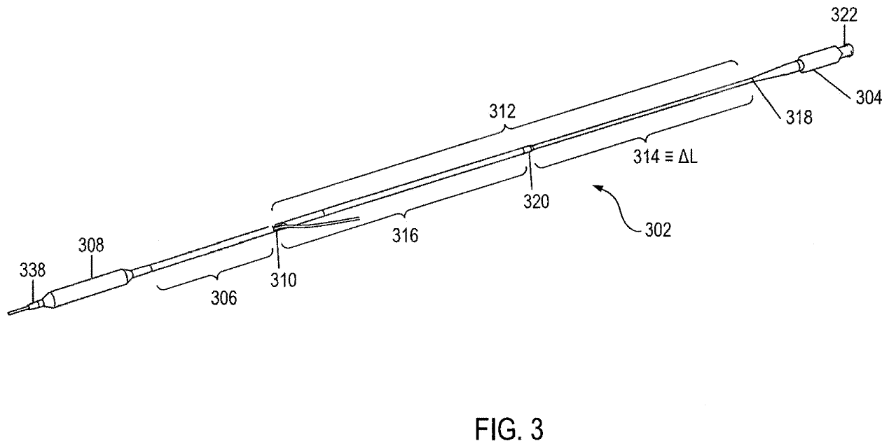

FIG. 3 shows a third implementation of the length-adjustable angioplasty balloon catheter. The third implementation is similar to the second implementation, discussed above with reference to FIGS. 2A-B. The third implementation of the length-adjustable angioplasty balloon catheter 302 features the smaller single-port mainfold 304 included in the second implementation. The third-implementation also includes a dual-lumen portion of the catheter shaft 306 between the inflatable catheter balloon 308 and the guide-wire port 310. However, unlike the second implementation, the third implementation includes a two-member single-lumen portion of the catheter shaft 312 that includes a smaller-outer-diameter portion 314 and a larger-outer-diameter portion 316. The smaller-outer-diameter portion 314 slides into the larger-outer-diameter portion 316 to provide length adjustability. In FIG. 3, a smaller-outer-diameter portion 314 is shown extended. When contracted, the tip of the kink-protection sleeve 318 is advanced toward a fluid-tight seal 320 as the smaller-outer-diameter portion 314 slides into the larger-outer-diameter portion 316. The fluid-impermeable seal 320 allows the smaller-outer-diameter portion 314 to slide into the larger-outer-diameter portion 316 while maintaining a fluid-impermeable inflation lumen extending from the inflation port 322 to the inflatable balloon catheter 308.

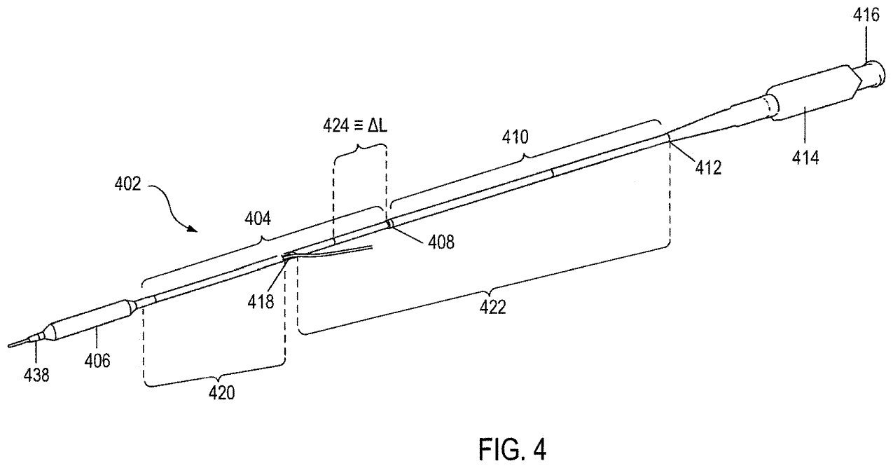

FIG. 4 illustrates a fourth implementation of the length-adjustable angioplasty balloon catheter. A fourth implementation 402 is similar to the third implementation, described above with reference to FIG. 3. However, in the fourth implementation, the portion of the catheter shaft 404 between the inflatable catheter balloon 406 and the fluid-impermeable fitting 408 slides into the portion of the catheter shaft 410 between the fluid-impermeable fitting 408 and the tip of the kink-protection sleeve 412. Thus, the first portion 410 of the catheter shaft has a larger inner diameter than the outer diameter of the second portion 404 of the catheter shaft. As with the implementation shown in FIG. 3, the manifold 414 has a single inflation port 416 and a guide-wire port 418 is located in the second portion 404 of the catheter shaft.

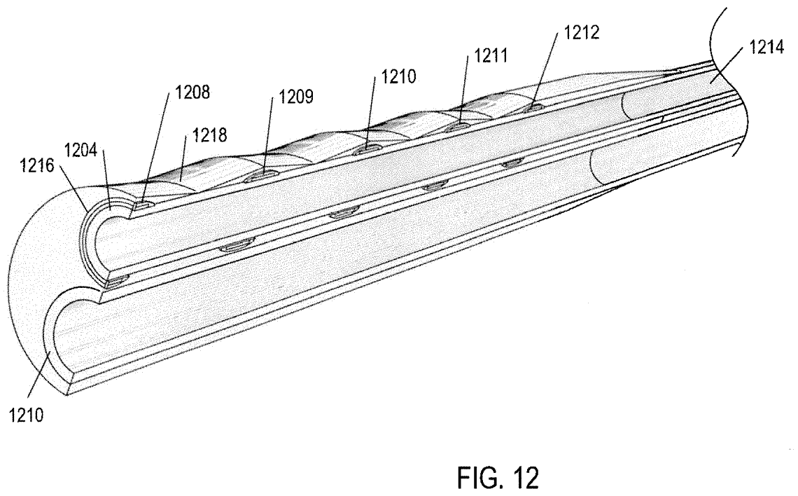

FIGS. 5A-B illustrate a fifth implementation of the length-adjustable angioplasty balloon catheter. A contracted configuration of the length-adjustable catheter is shown in FIG. 5A and an extended configuration of the length-adjustable catheter is shown in FIG. 58. The fifth implementation bears a similarity to the first implementation, discussed above with reference to FIGS. 1A-B. As in the first implementation, the fifth implementation includes a manifold 508 with an inflation port 510 and a guide-wire port 512. In a fifth implementation, a middle portion of the catheter shaft, 514 in FIG. 5A and 516 in FIG. 5B, comprises a dual-lumen section with an upper inflation lumen and a lower guide-wire lumen, similar to the variable portion of the catheter shaft of the first implementation discussed above with reference to FIGS. 1A-B. However, unlike the first implementation, the middle portion of the catheter shaft 514 and 516 in the fifth implementation is a rigid member over which a first sheath portion 520 and a second sheath portion 522 of the catheter shaft slides. The second sheath portion 522 of the catheter shaft includes two locking engagement zones 524 and 526 that provide fluid-impermeable seals for at least the inflation lumen, in a first set of implementations, and for both the inflation lumen and the guide-wire lumen, in a second set of implementations. These locking engagement zones are discussed further, below, with respect to FIG. 12. The middle section 514 and 516 is, in certain implementations, formed from a metal hypotube. In alternative implementations, the dual-lumen shaft sections may feature coaxial, nested lumens, with the inflation lumen surrounding the guide-wire lumen. The middle section may be fixedly attached to one of the sheath portions 520 and 522, in certain implementations, and is slidably attached to both the first 520 and second 522 sections in alternative implementations.

In all of the above-discussed implementations, the various components of the length-adjustable catheter may be composed of any of many different metals, metal alloys, polymeric materials, and composite materials, including fibers and particulate matter embedded in amorphous, non-crystalline, or semicrystalline micro-crystalline materials. By varying the material compositions of the components, length-adjustable catheters with different operational characteristics are implemented, including differences in rigidity and pliability, differences in the forces needed to extend and contract the variable-length portions of the catheter shaft, differences in surface characteristics with respect to fluids, gases, and biological environments in which the catheter is used, differences in the ability of various imaging technologies to locate and image portions of the catheter when inserted into a patient's blood vessels, and other such characteristics and parameters.

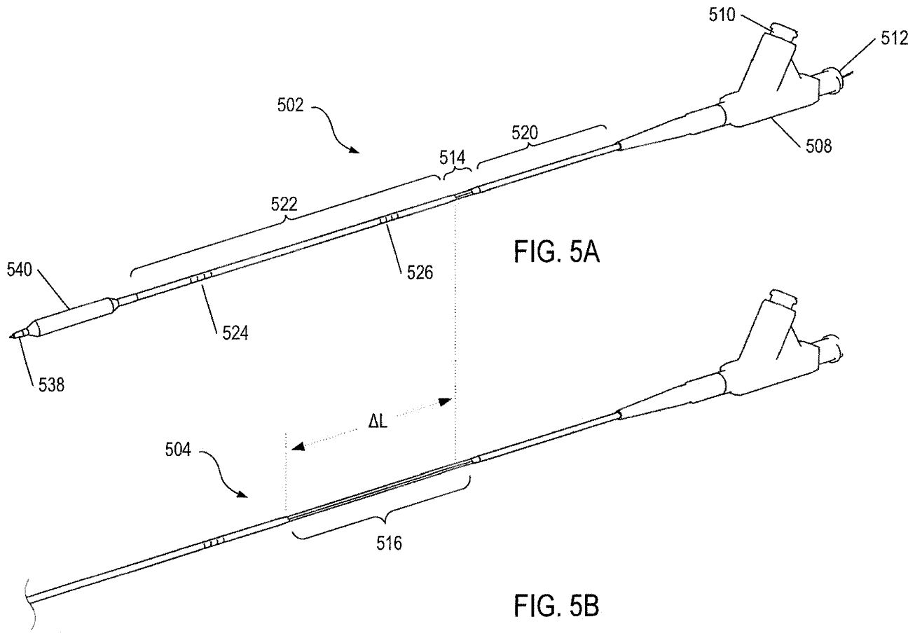

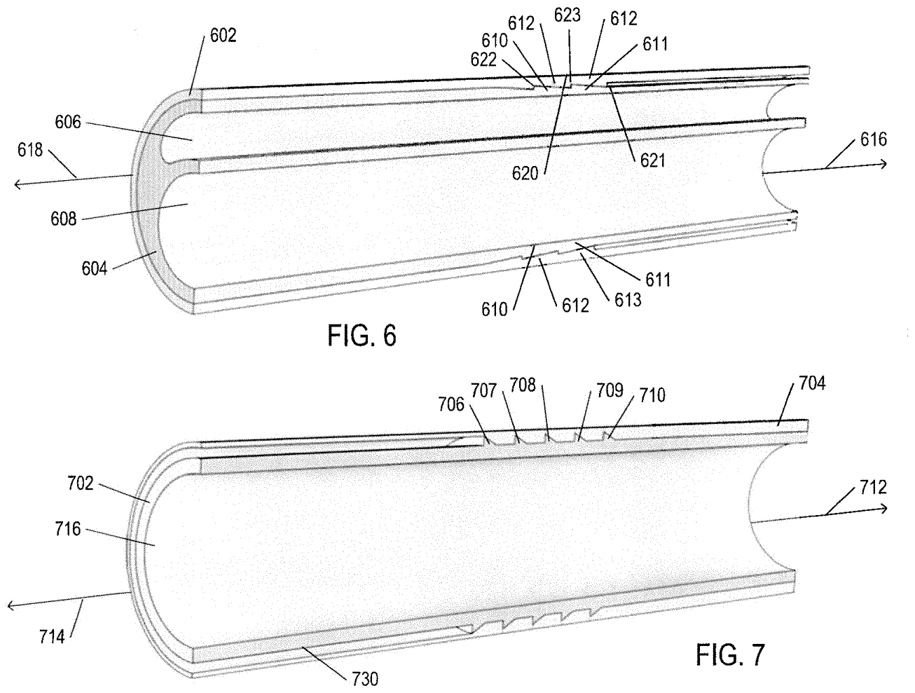

FIGS. 6-11 show a variety of different implementations of dual-lumen and single-lumen shaft-tube engagement configurations and locking mechanisms. These engagement configurations and locking mechanisms may be employed in various of the implementations of the length-adjustable angioplasty balloon catheter, discussed above with reference to FIGS. 1A-5B, as well as in various additional length-adjustable catheter implementations to facilitate engagement and mechanical locking of variable-length portions of the shaft of a length-adjustable catheter with respect to adjacent, enclosed, or enclosing catheter-shaft components.

FIG. 6 shows an outer shaft-member tube 602, in cross-section, in which an inner dual-lumen tube 604, also shown in cross-section, is slidably engaged. The inner dual-lumen tube includes an inflation lumen 606 and a wire-guide lumen 608. The inflation lumen and wire-guide lumen 606 and 608 run parallel within the inner tube and are each separately enclosed without fluid communication with one another. In the implementation shown in FIG. 6, the inner tube 604 includes several, annular, ramp-like features 610 and 611 on the outer external surface of the inner tube, shown in cross-section in FIG. 6. Complementary ramp-like features are inscribed within the inner surface of the outer tube 602 to form annular complementary ramp-like features. The annular ramp-like features on the external surface of the inner tube intermesh with the complementary annular ramp-like features inscribed within the inner surface of the outer tube 612 and 613 to lock the position of the inner tube with respect to the outer tube at the position shown in FIG. 6. Application of a longitudinal force to the inner tube 604 in the direction of arrow 616, a longitudinal force on the outer tube in the direction of arrow 618, or opposing longitudinal forces to the inner and outer tubes in the direction of arrows 616 and 618, can overcome forces approximately normal to the ramp-feature surfaces that hold the inner and outer tubes in the position illustrated in FIG. 6. resulting in relative movement of the inner tube with respect to the outer tube in the direction of arrow 616. This movement involves sliding of the ramp-like features on the outer surface of the inner tube against the ramp-like feature surfaces inscribed along the inner surface of the outer tube until the downward-pointing edges of the ramp-like features inscribed within the inner surface of the outer tube 620 and 621 slide past the tops of the ramp-like features 622 and 623 inscribed on the external surface of the inner tube. The tips of the ramp-like features inscribed on the inner surface of the outer tubes 620 and 621 then descend the steep, short faces of the ramp-like features inscribed on the outer surface of the inner tube to again lock into a stable position that represents an advancement of the inner tube in a direction of arrow 616 by a distance corresponding to one ramp-like feature with respect to the outer tube. Movement of the inner tube with respect to the outer tube in a direction opposite from arrow 616 is prevented by the steep, tooth-like notches of the complementary ramp-like features. When the inner tube is translated in the direction of arrow 616 with respect to the outer tube, sliding of the surfaces of the ramp-like features followed by the steep descent of the tips 620 and 621 of the ramp-like features inscribed in the inner surface of the outer tube to a next, locked position provide a haptic, tactile, or sensory signal to a treatment provider who is adjusting the length of a length-adjustable catheter. The dimensions of the complementary ramp-like features, as well as the dimensions of the inner and outer tubes and the inflation lumen and guide-wire lumens within the inner tube may all be varied to generate a variety of related implementations having different operation characteristics, including different incremental translations between locking positions, different amounts of forces needed to be applied in order to move the inner tube with respect to the outer tube, and other such operational characteristics. The inner and outer tubes may comprise any of a variety of different types of polymeric, metal, metal alloy, and other materials, which also impart variations in the operational characteristics of the length-adjustable catheter in which the engagement configuration and locking mechanism is employed.

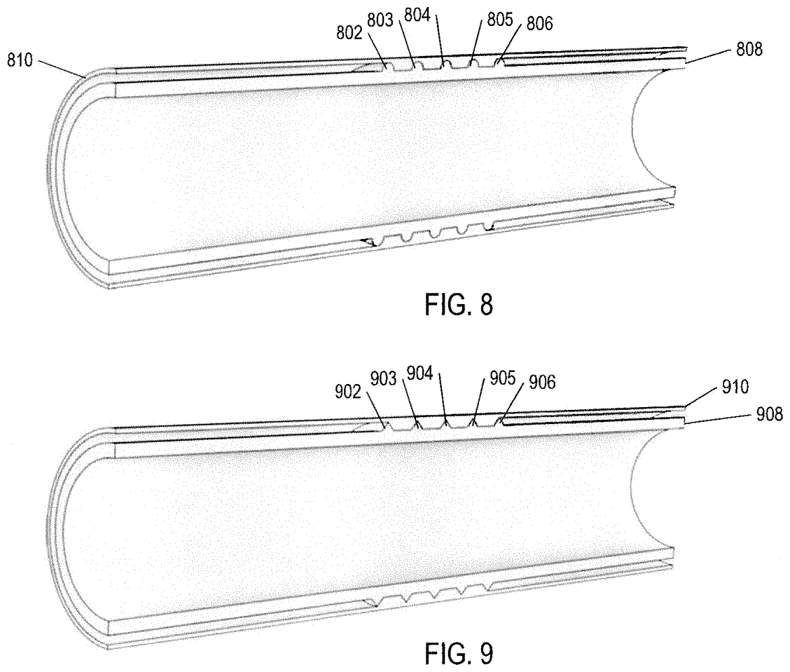

FIG. 7 illustrates another type of engagement configuration and locking mechanism used in various implementations of the above-described length-adjustable angioplasty balloon catheter. In FIG. 7, an inner, single-lumen tube 702, shown in cross-section, resides within a larger-diameter, outer catheter-shaft tube 704. Sawtooth-like features 706-710 are circularly disposed around the outer surface of the inner tube. As with the complementary ramp-like features discussed above with reference to FIG. 6, the sawtooth-like features on the outer surface of the inner tube, shown in cross-section in FIG. 7, fit within complementarily shaped grooves inscribed within the inner surface of the outer sheath or tube 704. As in the case in the implementation shown in FIG. 6, a longitudinal force applied in the direction of arrow 712 on the inner tube, in the direction of arrow 714 on the outer tube, or two opposing forces applied together to the inner and outer tubes in the directions 712 and 714, respectively, allow the inner tube to advance in the direction of arrow 712 with respect to the outer tube by an interval equal to the spacing between the sawtooth-like features. As in the implementation discussed above with reference to FIG. 6, the relative movement of the inner tube with respect to the outer tube provides haptic, tactile, or sensory feedback to a treatment provider extending or contracting a length-adjustable catheter. As with the implementation discussed above with reference to FIG. 6, the engagement configuration and locking mechanism illustrated in FIG. 7 allows for movement of the inner tube in the direction of arrow 712 with respect to the outer tube, but prevents movement of the inner tube with respect to the outer tube in the opposite direction. As with the implementation discussed above with reference to FIG. 6, and subsequent implementations discussed below with reference to FIGS. 8-11, the operational characteristics of the engagement configuration and locking mechanism illustrated in FIG. 7 may vary as the material composition, dimensions, inter-feature spacings, and other parameters of the engagement configuration and locking mechanism are varied.

FIGS. 8-9 illustrate alternative engagement configurations and locking mechanisms, similar to those discussed above with reference to FIG. 7. As shown in FIG. 8, circular, rib-like features 802-806, shown in cross-section in FIG. 8, are formed on the external surface of the inner tube 808 and fit into complementary circular, rounded well-like features inscribed on the inner surface of an outer tube 810. As with the engagement configuration and locking mechanism discussed above with reference to FIG. 7, the complementary features hold the inner tube at a stable position with respect to the outer tube. As shown in FIG. 9, symmetrical, wedge-shaped annular features 902-906 are formed in the outer surface of the inner tube 908 complementary to V-shaped annular depressions on the inner surface of an outer tube 910. In certain implementations of the engagement configuration and locking mechanism shown in FIGS. 8 and 9, the locking mechanisms provide advancement of the inner tube with respect to the outer tube in both longitudinal directions. In other implementations, a stop feature may be included at one extremity of the annular features on the external surface of the inner tube or the annular features on the inner surface of the outer tube, or both, to prevent advancement in one direction past the stop position but allow advancement in the other direction.

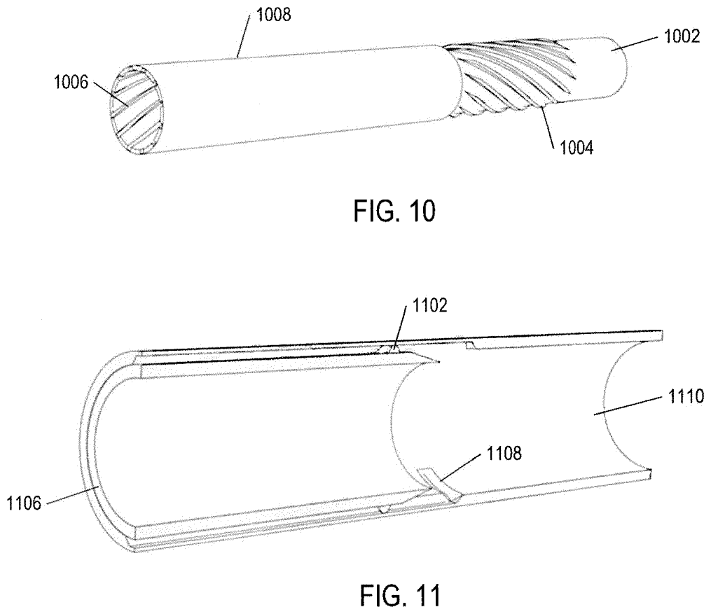

FIG. 10 illustrates an additional implementation of an engagement configuration and locking mechanism. In this implementation, the external surface of an inner tube 1002 includes helical thread-like features, including helical thread-like feature 1004, that are complementary to helical depressions or wells, such as helical depression 1006, in the inner surface of an outer tube 1008. The helical threads and/or notches may span only a portion of the outer surface of the inner tube and inner surface of the outer tube, providing for relative motion of the inner tube with respect to the outer tube over only that distance along the lengths of the two tubes. Alternatively, annular non-notched sections of the inner surface of the outer tube, positioned at intervals along the length of the outer tube, can provide regularly spaced stopping points, as in the implementations discussed above with reference to FIGS. 6-9, to allow the inner tube to be advanced with respect to the outer tube by fixed increments. In additional implementations, the inner surface of the outer tube may have raised threads complementary to wells or notches in the external surface of the inner tube.

FIG. 11 provides yet an additional implementation of an engagement configuration and locking mechanism. As shown in FIG. 11, rounded or wedge-shaped pins 1102 and 1104 formed on the outer surface of the inner tube 1106 are complementary to curved wells or notches 1108 on the inner surface of the outer tube 1110. The pins 1102 and 1104 can be rotated, by rotation of the inner tube relative to the outer tube, to engage and disengage from the notches 1108. In this implementation, rotation of the inner tube to disengage the pins from the notches followed by application of a longitudinal force to either or both the inner and outer tubes allows for movement of the inner tube with respect to the outer tube in the longitudinal direction. Following translation of the inner tube with respect to the outer tube, the inner tube can be again rotated in order to engage the pins in a next slot within the inner surface of the outer tube to lock the position of the inner tube with respect to the outer tube.

FIG. 12 illustrates an engagement configuration and locking mechanism that, as one example, may be employed in the length-adjustable angioplasty balloon catheter implementation shown in FIGS. 5A-B. In this implementation, a double-lumen pair of inner tubes 1202 and 1204 are enclosed within an outer sheath or tube 1206. Annular radio-opaque marker rings 1208-1212 are positioned at regular intervals along the outer surface of the inflation lumen 1214 of the double-lumen inner tube. The radio-opaque marker rings are each encapsulated within an elastomeric sealant ring, such as the elastomeric sealant ring 1216 cove the need nular radio-opaque marker 1208. The elastomeric sealant rings and covered radio-opaque marker rings together form annular protrusions, or bumps, at regular intervals on the external surface of the inflation lumen 1214. These annular bumps are complementary to rounded wells within the inner surface of the outer sheath 1206. The outer sheath has a series of annular grooves, such as annular groove 1218, patterned along the external surface of the sheath to facilitate manipulation by a treatment provider. The inner double-lumen tube can be incrementally translated with respect to the outer sheath 1206 by incremental lengths equal to the distance between the elastomeric annular sealant rings and complementary annular wells along the outer surface of the inner tube and interior surface of the outer sheath.

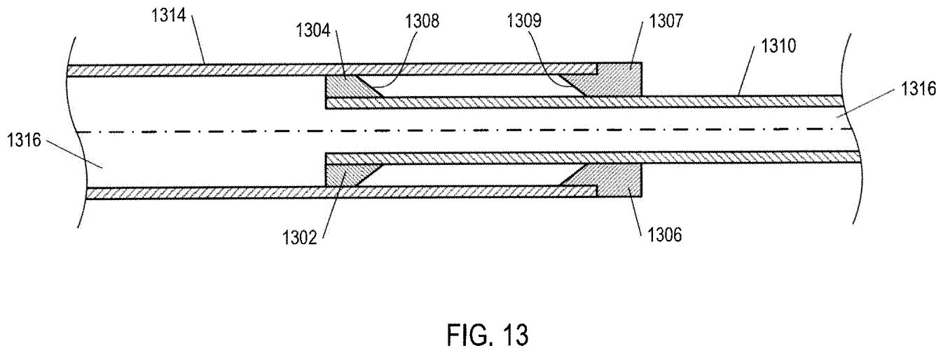

The length-adjustable angioplasty balloon catheter implementations discussed above with reference to FIGS. 3-4 include a slidable length adjustment in which a larger-diameter portion of the catheter shaft slides over a smaller-diameter portion of the catheter shaft. FIG. 13 shows, in cross-section, a fitting that provides a fluid-impermeable slidable seal to facilitate sliding of a smaller-diameter portion of a length-adjustable catheter shaft within a larger-diameter portion of the catheter shaft. As shown in FIG. 13, the fitting includes an inner sealing annulus 1302 and 1304, shown in cross-section in FIG. 13, and a complementary outer sealing ring 1306 and 1307. The sealing rings have complementary ramp-like surfaces, including ramp-like surfaces 1308 and 1309, so that the two sealing rings together form a stop when the smaller-diameter portion of the catheter shaft 1310 is translated in the direction of arrow 1312 with respect to the larger-diameter portion of the catheter shaft 1314 and reaches a point where the ramp-like surface of the inner sealing ring 1308 meets the complementary ramp-like surface 1309 of the outer sealing ring 1307. The inner and outer sealing rings together provide a fluid-impermeable fitting that slidably connects the smaller-diameter portion of the catheter shaft 1310 with the larger-diameter portion of the catheter shaft 1314 to provide a single, continuous lumen 1316 that is length-adjustable.

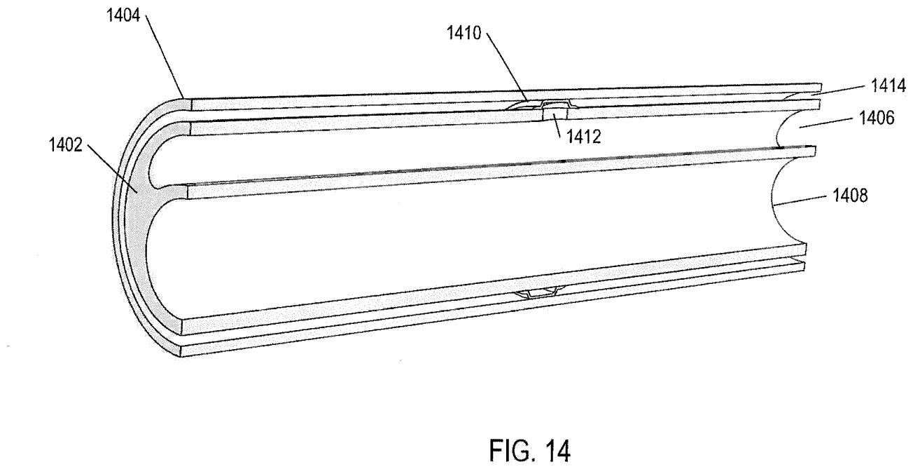

FIG. 14 shows an additional engagement configuration and locking mechanism. As shown in FIG. 14, a double-lumen inner tube 1402 is slidably mounted within an outer, larger-diameter tube 1404. The double-lumen tube includes an inflation lumen 1406 and a guide-wire lumen 1408. An annular elastomeric membrane 1410 is affixed to the outer surface of the inner tube 1402 above an inflation port 1412. The inflation port, when pressure within the inflation lumen 1406 exceeds a first threshold pressure, expands into the elastomeric membrane 1410 to mechanically engage and lock the position of the inner tube with respect to the outer tube. In addition, the pressure-induced expansion of the elastomeric membrane 1410 creates a fluid-impermeable seal that seals the narrow lumen 1414 between the outer tube 1404 and inner tube 1402. The first pressure threshold is significantly less than a second pressure threshold, above which the balloon catheter of a length-adjustable angioplasty balloon catheter is inflated via pressure within the inflation lumen 1406.

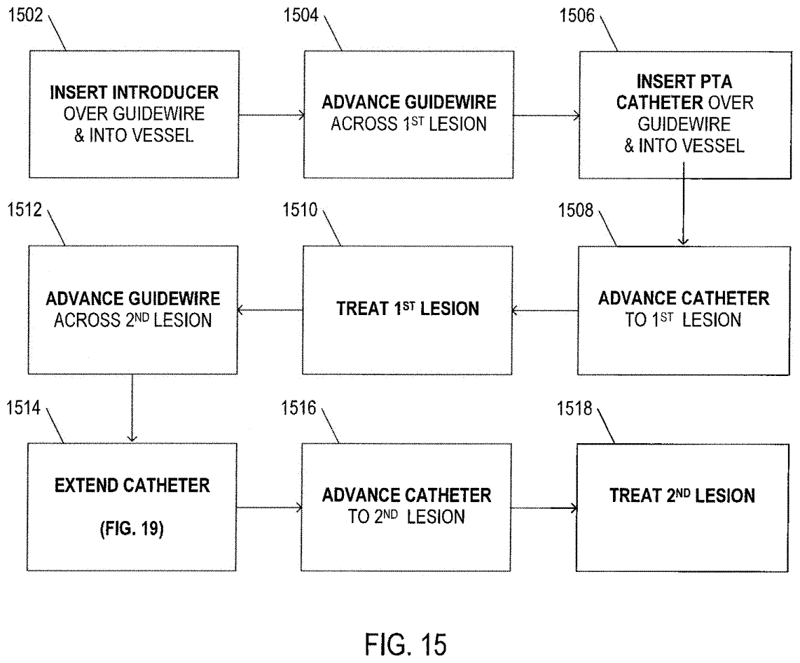

FIG. 15 shows a series of steps carried out by a treatment provider, using a length-adjustable angioplasty balloon catheter, to treat multiple blockages or lesions within a patient's blood vessel. In step 1502, the treatment provider inserts an introducer sheath into the patient's blood vessel under hemostatic conditions to provide access to the blood vessel for catheters and other treatment devices. In step 1504, the treatment provider advances a guide wire through the introducer sheath into the patient's blood vessel and positions the end of the guide wire within a first target treatment area. In step 1506, the treatment provider inserts a length-adjustable angioplasty balloon catheter through the introducer sheath and over the guide wire and, in step 1508, advances the length-adjustable catheter along the guide wire to a position at which the length-adjustable catheter can be manipulated to treat or ameliorate the blockage or lesion and restore luminal patency. As the result of treatment at the first target site, additional lesions further down the patient's blood vessel may become observable. In step 1512, the treatment provider advances the guide wire to a next target treatment site. In step 1514, the treatment provider extends the length-adjustable catheter and, in step 1516, advances the end of the length-adjustable catheter to the second target site. In step 1518, the catheter is manipulated in order to remove or ameliorate a second blockage or lesion. The steps shown in FIG. 15 are an example of the use of a length-adjustable catheter. Other examples include extending a length-adjustable catheter or contracting a length-adjustable catheter, following initial positioning, in order to correct a discrepancy in the initial positioning of the catheter. Additional examples include treatment of more than two lesions or blockages.

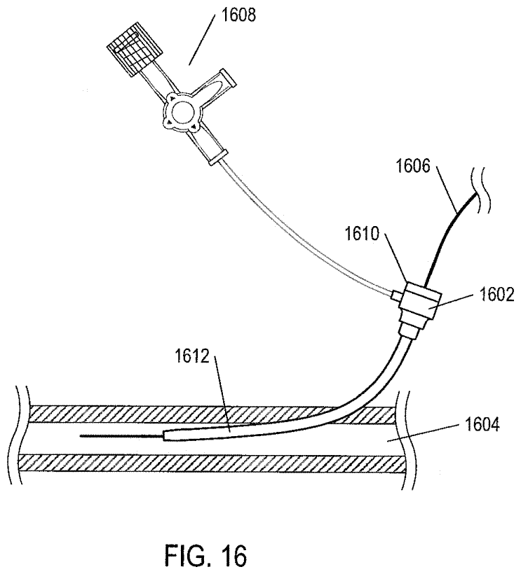

FIGS. 16-20 provide additional illustration of selected steps discussed above with reference to FIG. 15. FIG. 16 illustrates insertion of an introducer sheath into a patient's blood vessel followed by insertion of a guide wire. The introducer 1602 is inserted through a puncture site in the wall of the patient's blood vessel 1604. The introducer, in the example shown in FIG. 16, includes a three-way stopcock valve 1608 for delivery of fluids into the patient's blood vessel and a hemostasis value 1610 for prevention of bleeding. The introducer sheath 1612 provides for insertion and guidance of guide wires and subsequent insertion of catheters over the guide wires under hemostatic conditions.

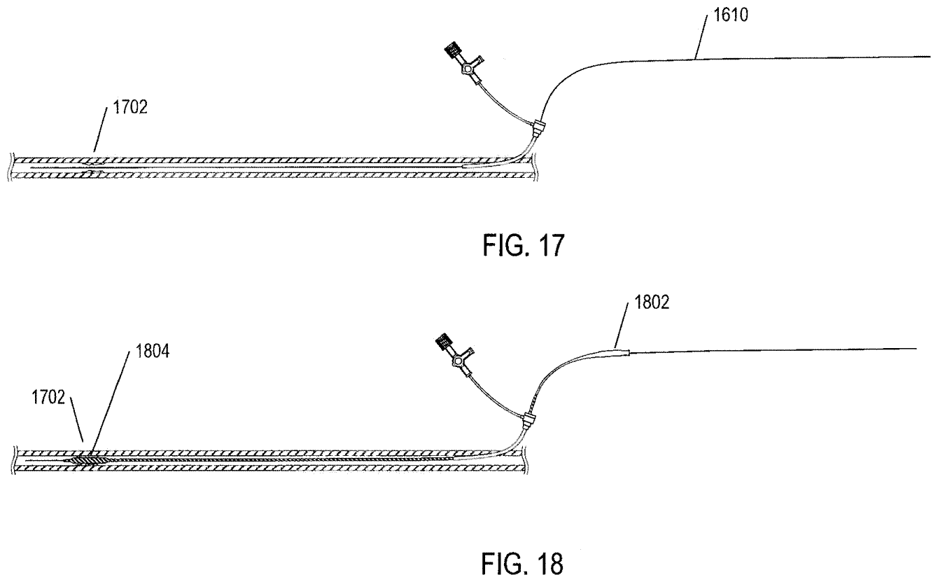

FIG. 17 illustrates advancement of a guide wire into a patient's blood vessel across a target treatment site. The tip of the guide wire 1610 has been advanced, relative to the initial position shown in FIG. 16, past a first treatment site 1702. Advancement of the guide wire may be monitored by radiographic means or other medical-imaging technology.

FIG. 18 illustrates introduction of a length-adjustable catheter over a guide wire through an introducer into a patient's blood vessel. The length-adjustable catheter 1802 has been positioned so that the catheter balloon 1804 straddles the treatment target site 1702. The catheter balloon can be subsequently inflated, via application of fluid under pressure through the inflation lumen, in order to ameliorate the blockage or lesion at the treatment sites. Alternatively, the length-adjustable catheter can be manipulated, in other ways, to ameliorate or dislodge an obstruction or blockage.

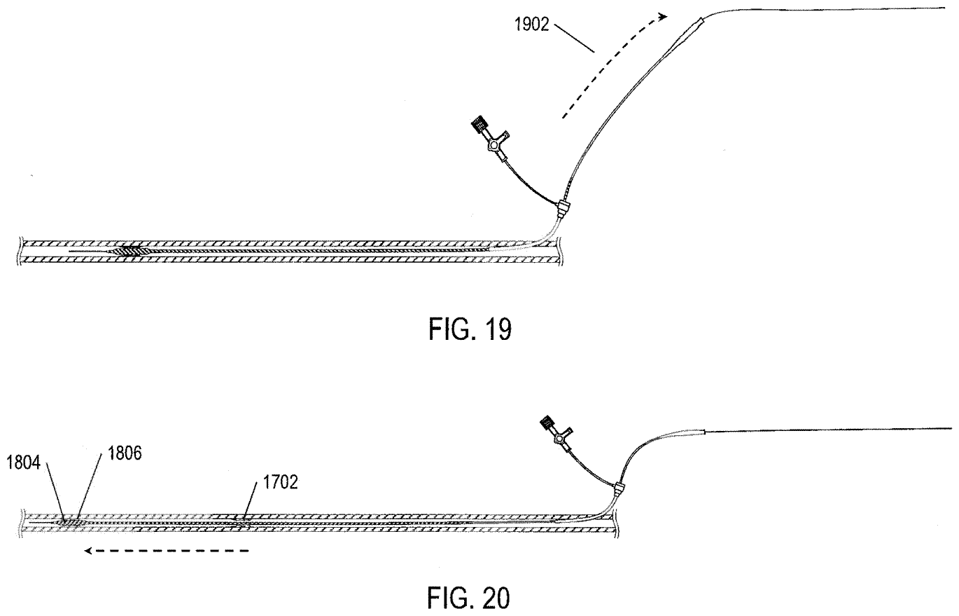

FIG. 19 illustrates length-adjustable catheter extension. As indicated by dashed arrow 1902, a treatment provider manipulates the catheter shaft that is exposed above the introducer in order to lengthen the catheter, with the lengthening occurring in the portion of the catheter shaft external to the introducer and patient's blood vessel. Various different techniques may be used for catheter extension, depending on the types of variable-length portions of the catheter shaft and the types of engagement configurations and locking mechanisms included in the catheter. FIG. 20 illustrates advancement of an extended length-adjustable catheter to a second target area. The extended length-adjustable catheter is advanced along the guide wire past the first target site so that the catheter balloon 1804 is positioned to straddle a second treatment site 1806.

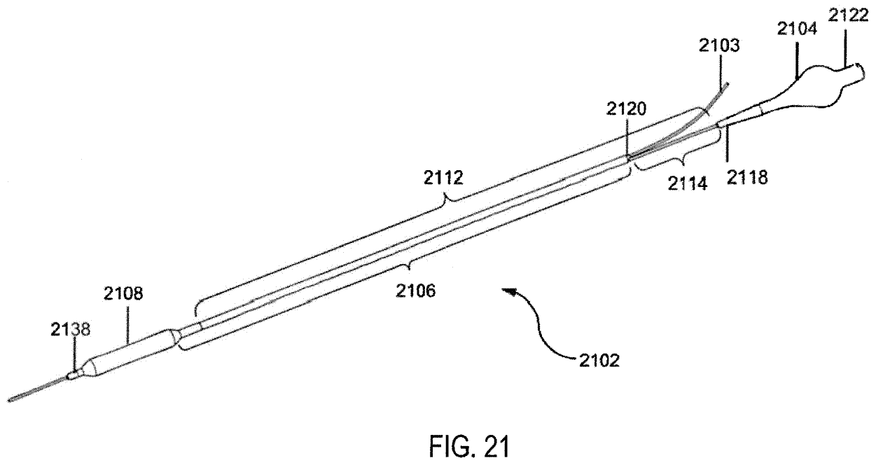

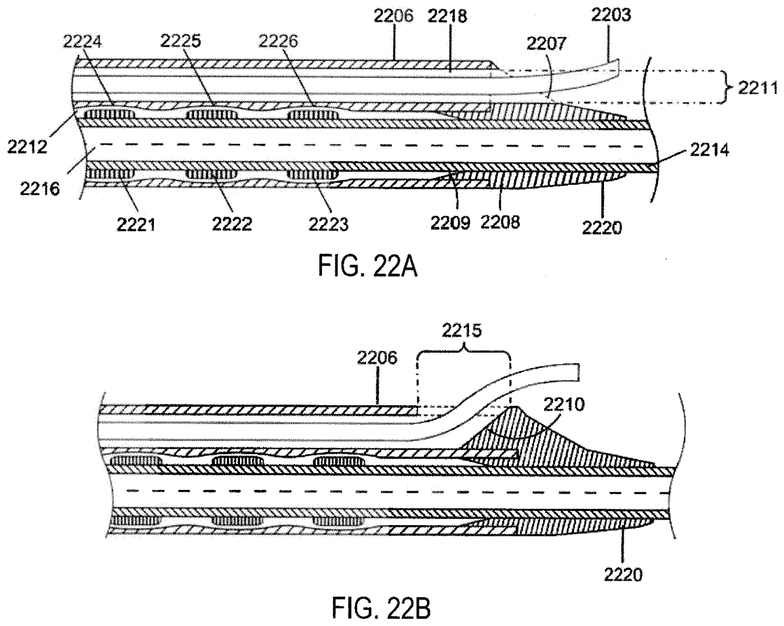

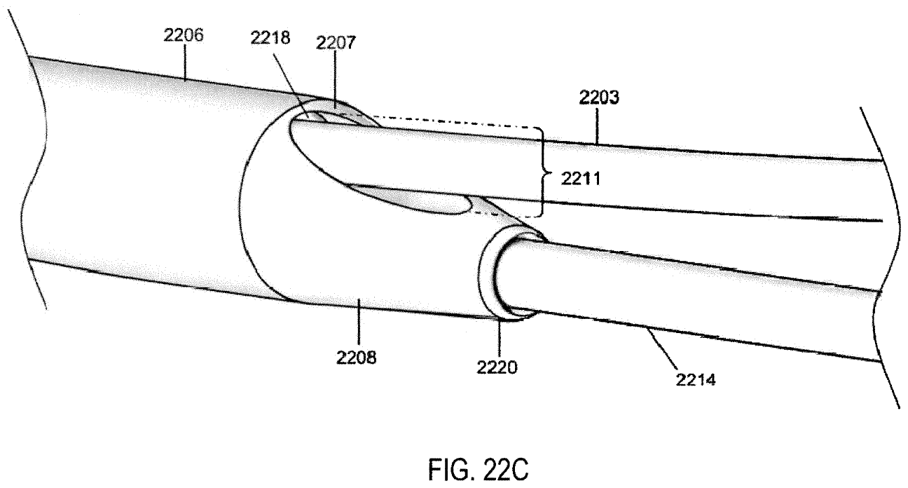

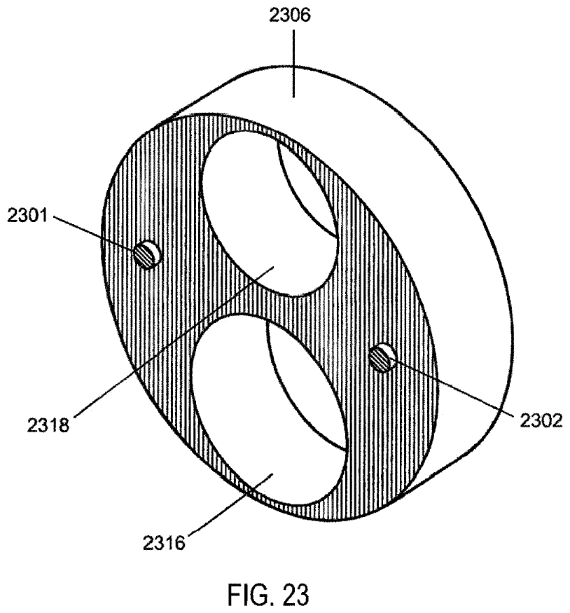

FIG. 21 shows a sixth implementation of the length-adjustable angioplasty balloon catheter. The sixth implementation is similar to the third implementation, discussed above with reference to FIG. 3. The sixth implementation of the length-adjustable angioplasty balloon catheter 2102 features a single-port manifold 2104 similar to the manifold included in the third implementation. The sixth implementation also includes a two-member catheter shaft 2112 that comprises a larger-outer-diameter portion 2106, configured as a dual-lumen tube, and a smaller-outer-diameter portion 2114, configured as a single lumen tube. However, unlike the third implementation, the guide wire port 2120 is now located at the proximal end of the larger-outer-diameter portion 2106 of the two-member catheter shaft, rather than at an intermediate or distal position within the catheter shaft, as it is in the third implementation, discussed above with reference to FIG. 3. As a result, the dual-lumen portion now fully extends along the complete length of the larger-outer-diameter portion, from between the inflatable catheter balloon 2108 and the guide-wire port 2120. In addition, the guide wire 2103 does not exit at an angle from a lateral surface of the larger-outer-diameter portion, as shown in FIG. 3, but instead exits in parallel to the smaller-outer-diameter portion 2114 of the catheter shaft 2112. Further, the guide-wire port 2120 incorporates a fluid-impermeable seal that terminates both the guide-wire lumen and the inflation lumen of the dual-lumen portion 2106 of the catheter shaft. The smaller-outer-diameter, single lumen portion 2114 slides into the larger-outer-diameter, dual-lumen portion 2106 to provide length adjustability. In FIG. 21, the smaller-outer-diameter portion 2114 is shown contracted. When contracted, the tip of the kink-protection sleeve 2118 is advanced toward the guide wire port 2120 that includes the fluid-tight seal as the smaller-outer-diameter portion 2114 slides into the larger-outer-diameter portion 2106. The fluid-impermeable seal allows the smaller-outer-diameter portion 2114 to slide into the larger-outer-diameter portion 2116 while maintaining a fluid-impermeable inflation lumen extending from the inflation port 2122 to the inflatable balloon catheter 2108. The impermeable seal is explained, below, with reference to FIGS. 22A and 22B.

FIGS. 1A-1B, and FIGS. 5A-5B feature over-the-wire catheters, while FIGS. 2A-2B, and FIGS. 3-4 feature rapid-exchange catheters. The guide wire functionality of the length-adjustable angioplasty balloon catheter 2102 of FIG. 21 differs from the previously described embodiments by allowing the use of the larger-outer-diameter portion 2106 as an over-the-wire catheter, and, when length-adjusting the catheter shaft 2112 through deployment of the smaller-outer-diameter portion 2114, allowing the use of the catheter as a rapid-exchange catheter. The length-adjustable angioplasty balloon catheter 2102 therefore is configured as a dual-use, over-the-wire-and-rapid-exchange catheter.

FIGS. 22A-C show, in cross-section, two fitting configurations that describe the fluid-impermeable seal introduced in FIG. 21 and an additional three-dimensional representation of the configuration shown in FIG. 22A. FIGS. 22A-B show, in cross-section, a fitting that provides a fluid-impermeable seal to facilitate sliding of a smaller-outer-diameter portion 2214 of a length-adjustable catheter shaft within a larger-outer-diameter portion 2206 of the catheter shaft. As shown in FIG. 22A, the fitting includes an asymmetrical and cone-shaped sealing plug 2220. The sealing plug includes an upper portion 2207 that tapers towards the proximal end of the larger-outer-diameter portion 2206, and provides an annular, straight-ended orifice 2211 that terminates the guide wire lumen 2218. The lower portion 2208 includes an inner annulus with a ramp-like surface 2209 that is fused with the larger-outer-diameter portion 2206 of the catheter shaft. The lower portion is formed around the smaller-outer-diameter portion 2214 to provide a fluid-tight seal of the inflation lumen 2212 surrounding the smaller-outer-diameter portion 2214. The inflation lumen 2212 provides a fluid connection to the inflation lumen 2216 contained within the smaller-outer-diameter portion (distal connection not shown). The exterior surface of the smaller-outer-diameter portion 2214 and the interior surface of the lower portion 2208 of the sealing plug 2220 together provide a fluid-impermeable fitting that slidably connects the smaller-diameter portion of the catheter shaft with the larger-diameter portion of the catheter shaft to provide a single, continuous inflation lumen that is length adjustable. The exterior surfaces of the fitting are shown in a three-dimensional representation in FIG. 23C, using the same numerical labels used in FIG. 22A. FIG. 22B shows an alternative guide wire exit port configuration. In FIG. 22B, the upper portion of the sealing plug 2220 includes a guide wire ramp 2210 so that the guide wire can exit at an angle from a lateral surface of the larger-outer-diameter portion 2206 by passing through an annular, lateral orifice 2215 of the catheter shaft. As a result, when the guide wire is passed over the angled ramp, the guidewire can still proceed in parallel to the smaller diameter portion 2214 of the catheter shaft, as described in FIG. 21, but at a comparatively larger distance. This configuration enables the treatment provider to more easily place his fingers between the guide wire and the catheter shaft for ease of handling.

As another embodiment, FIGS. 22A and 22B feature annular radio-opaque marker rings 2221-2223 positioned at regular intervals along the outer surface of a single-lumen inner tube that forms the smaller-outer-diameter portion 2214 of a length-adjustable catheter shaft. The radiopaque marker rings form annular protrusions at regular intervals on an external surface of the smaller-outer-diameter portion, and are complementary to rounded wells 2224-2226 present on an inner surface of a dual-lumen outer tube that forms the larger-outer-diameter portion 2206. The single-lumen inner tube can be incrementally translated with respect to the dual-lumen outer tube by lengths equal to the distance between the annular marker rings and complementary annular wells along the exterior surface of the single-lumen inner tube and interior surface of the dual-lumen outer tube to facilitate manipulation by a treatment provider. The most proximal radiopaque marker ring 2223 forms a stop when the smaller-diameter portion of the catheter shaft 2214 is translated proximal with respect to the larger-diameter portion 2206 of the catheter shaft and reaches a point where the ramp-like surface of the inner annulus 2209 meets the surface of the outer radiopaque marker ring 2223. When the annular marker rings are slid over the complementary annular wells, tactile feedback is provided to a treatment provider, so that the treatment provider can determine an amount of length adjustment. Simultaneously, or alternatively, the sealing plug can be manufactured from a radiopaque material, so that the treatment provider can angiographically determine a relative amount of length adjustment obtained between the sealing plug and the set of radiopaque marker rings.

FIG. 23 illustrates a dual-lumen reinforced shaft configuration adaptable to the currently disclosed length-adjustable catheters. FIG. 23 shows, in cross-section, a thin, disk-shaped portion of a dual-lumen shaft 2306, or shaft section, that includes an inflation lumen 2316, a guide wire lumen 2318, and a plurality of reinforcing threads 2301 and 2302 embedded along a length axis of the dual-lumen shaft. The reinforcing threads stabilize the dual-lumen configuration to provide greater torsional and bending stability than provided by a non-reinforced shaft. Further, mechanical translation is facilitated and kink resistance is increased by the reinforcement. The two or more threads can be arranged in both axial and non-axial orientations, as separate threads or overlapping with one another. In addition, the reinforcing threads may proceed in parallel, or cross at an angle to one another. The distance between neighboring threads, or threading density, as well as the crossing angle and crossings per distance can be suitably adapted to reinforce or stiffen the shaft to variable degrees at select locations of the catheter shaft. Suitable materials include fibers or filaments formed from metal and/or polymeric materials.

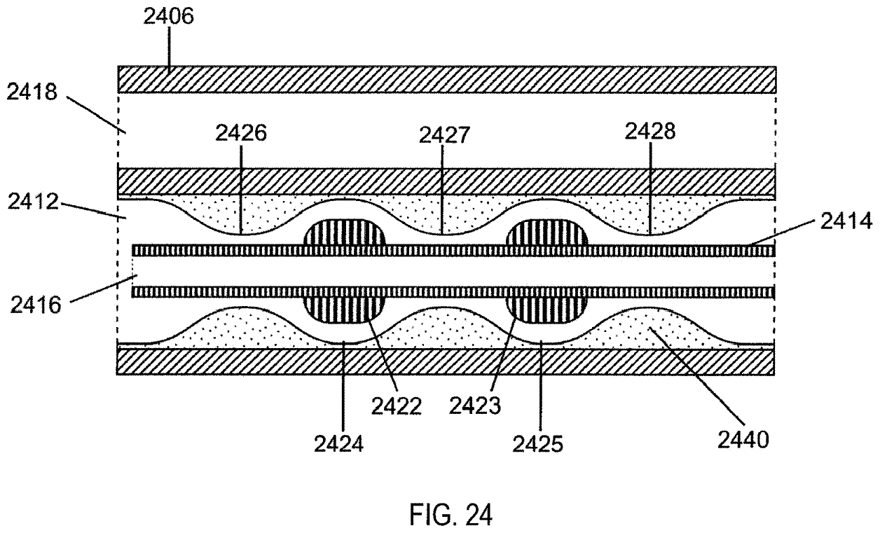

FIG. 24 shows an engagement, locking and position-indicating configuration that, as one example, may be employed in the length-adjustable angioplasty balloon catheter implementation shown in FIG. 21. In FIG. 24, annular radio-opaque marker rings 2423 and 2424, similar to those of FIG. 22A-B, are positioned at regular intervals along the outer surface of a single-lumen inner tube that forms the smaller-outer-diameter portion 2414 of a length-adjustable catheter shaft. The radiopaque marker rings form annular protrusions at regular intervals on an external surface of the smaller-outer-diameter portion, and are complementary to rounded wells 2422-2425 on an inner surface of a dual-lumen outer tube that forms the larger-outer-diameter portion 2406. However, unlike the implementation shown in FIG. 22, the series of annular protrusions 2426-2428 and rounded wells 2424-2425 are formed as a separate sealing, locking, and position-indicating layer 2440 that faces towards the inflation lumen 2412 and is joined with the inner surface of the dual-lumen outer tube. The layer 2440 is manufactured from an elastomeric sealant polymer imbued with radiopaque material. The sealant polymer may be formed from soft polymers of Shore hardness 60A or below, including polyvinyl rubbers, silicone, polyurethane, santoprene elastomers, and/or equivalents. Radiopacity is obtained by compounding the sealant polymer with radiopaque filler materials that include micronized barium sulfate or tungsten powder. The desired level of radiopacity of the sealant polymer can be controlled by adding the radiopaque filler material at weight proportions relative to the sealant polymer that range from about between 5-60 weight percent, or more preferably from about 20-40 weight percent. When the annular marker rings of the smaller-diameter inner tube are slid over the complementary annular wells of the larger-diameter outer tube, the treatment provider can angiographically determine the position of the set of radiopaque marker rings relative to the position of the sealing, locking and position-indicating layer 2440 to determine an amount of length adjustment. The soft sealant layer facilitates arresting the position of the radiopaque marker rings when the annular protrusions 2422 and 2423 are superimposed by translation against the annular protrusions 2427-2428 of the sealing layer, and provides for a fluid-impermeable seal when the protrusions are in direct contact with each other. When the annular marker rings are slid over the complementary annular wells, tactile feedback is observable by a treatment provider, so that the treatment provider can determine an amount of length adjustment.

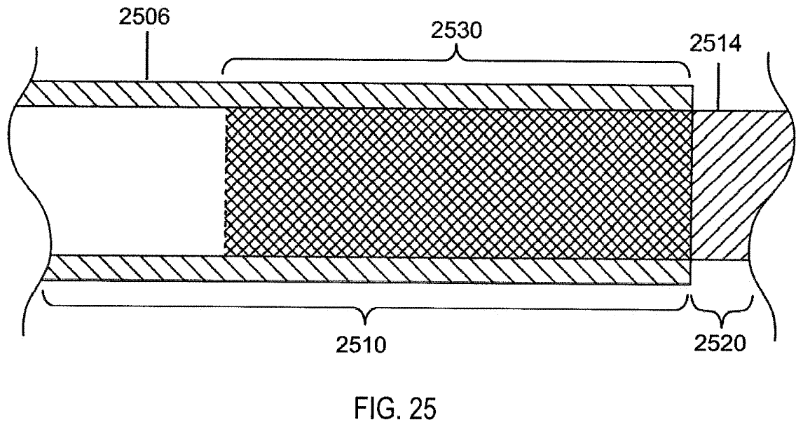

FIG. 25 illustrates a position-indicating configuration adaptable to the currently disclosed length-adjustable catheters. In FIG. 25, a smaller-diameter inner tube 2514 is shown inserted into a larger-diameter outer tube 2506. The smaller-diameter tube is formed from a material having a first radiopacity 2520, while the larger-diameter tube is formed from a material having a second radiopacity 2510. When the smaller diameter tube is slid into the larger-diameter tube, the radiopacities 2510 and 2520 of the materials forming the respective tubes combine, creating a section having a third radiopacity 2530. As a result, the treatment provider can angiographically determine the insertion length of a smaller-diameter tube slid into a larger-diameter tube of the currently disclosed length-adjustable catheters, which provides a method for determining continuous adjustment lengths that does not rely on of the above-discussed radiopaque marker rings.

Although the present invention has been described in terms of particular embodiments, it is not intended that the invention be limited to these embodiments. Modifications within the spirit of the invention will be apparent to those skilled in the art. For example, as mentioned above, there are many different possible implementations of a length-adjustable catheter. In general, the length of the catheter shaft can be altered, before and/or during a treatment procedure, in order to provide access to multiple positions within a blood vessel. In either the dual-lumen or single-lumen implementations, the inflation lumen remains fluid impermeable before, during, and after extension or contraction of the length-adjustable catheter. Furthermore, the catheter retains a proper balance between rigidity and flexibility, over the adjustable length range, for carrying out intended medical procedures. The various components of a length-adjustable catheter can be fabricated from a variety of different materials, including metals, metal alloys, polymeric materials, and other suitable material compositions. Length-adjustable catheters can be designed and manufactured to have a variety of different length ranges, diameters, internal lumen volumes, extension and contraction forces, surface characteristics, and other such characteristics. In many implementations, the length-adjustable catheters include engagement-configuration and locking mechanisms so that the medical practitioner can sense precise amounts of length adjustment and can lock the length-adjustable components of the catheter sheath at a fixed, intended length. In addition, various features may be provided at regular intervals along the catheter sheath to aid in catheter positioning, position sensing, and length adjustment, including optically detectable features and features detectable by various medical-imaging technologies.

It is appreciated that the previous description of the disclosed embodiments is provided to enable any person skilled in the art to make or use the present disclosure. Various modifications to these embodiments will be readily apparent to those skilled in the art, and the generic principles defined herein may be applied to other embodiments without departing from the spirit or scope of the disclosure. Thus, the present disclosure is not intended to be limited to the embodiments shown herein but is to be accorded the widest scope consistent with the principles and novel features disclosed herein.

1. A length-adjustable catheter comprising:
a shaft having
a first, proximal end,
a second, distal end,
a first, fixed-length portion that includes the first, proximal end,
a second fixed-length portion that includes the second, distal end,
a length-varying component between the first and second fixed-length portions, and
one or more locking features;
a manifold having
a first, distal end mounted over the first, proximal end of the shaft,
a second, proximal end, and
a diameter greater than a diameter of any shaft portion and component;
a guide-wire port that extends from the second, proximal end of the manifold;
a guide-wire lumen that extends, through the shaft, from the guide-wire port to an opening at the second, distal end of the shaft, a linear length axis of the guide-wire lumen parallel to a length axis of the shaft;
an inflation port that extends from the manifold at an acute angle with respect to the length axis of the shaft; and
an inflation lumen that extends, through the shaft, from the inflation port to an inflatable component, a length axis of a linear portion of the inflation lumen within the shaft parallel to the length axis of the shaft.

2. The length-adjustable catheter of claim I wherein the length-varying component is one or more of:
a longitudinally and plastically deformable dual-lumen shaft portion, having a folding pattern embedded into, or imprinted or molded on, the outer surface to facilitate longitudinal extension and contraction;
one or more fluid-impermeable seals that allow the first, fixed-length portion portion to slide relative to the second, fixed-length portion while maintaining fluid-impermeability of both the inflation lumen and the guide-wire lumen; and
a third fixed-length length portion and two or more fluid-impermeable seals that allow the first, fixed-length portion and the second, fixed-length portion to slide relative to the third fixed length portion while maintaining fluid-impermeability of both the inflation lumen and the guide-wire lumen.

3. The length-adjustable catheter of claim 2 wherein the one or more fluid-impermeable seals each comprises:
an inner sealing annulus on the outer surface of a first shaft portion; and
a complementary outer sealing ring with complementary ramp-like surfaces on the inner surface a second shaft portion.

4. The length-adjustable catheter of claim 2 wherein the one or more fluid-impermeable seals each comprises:
an annular elastomeric membrane affixed to the outer surface of a first shaft portion that expands against an inner surface of a second shaft portion.

5. The length-adjustable catheter of claim 2 wherein shaft portions that slide relative to one another have different diameters.

6. The length-adjustable catheter of claim 1 wherein the inflation lumen and guide-wire lumen are separate lumens, with no fluid communication between the inflation lumen and guide-wire lumen.

7. The length-adjustable catheter of claim 1 wherein each of the one or more locking features comprises one of:
annular, ramp-like features on the outer external surface of a first shaft portion and complementary ramp-like features inscribed within an inner surface of a second shaft portion;
sawtooth-like features circularly disposed around the outer surface of a first shaft portion and complementarily shaped grooves inscribed within an inner surface of a second shaft portion;
circular, rib-like features circularly disposed around the outer surface of a first shaft portion and complementarily circular, rounded, features inscribed within an inner surface of a second shaft portion;
wedge-shaped annular features disposed around the outer surface of a first shaft portion and complementarily V-shaped annular depressions within an inner surface of a second shaft portion:
helical thread-like features circularly on the outer surface of a first shaft portion and complementarily helical depressions or wells within an inner surface of a second shaft portion; and wedge-shaped pins on the outer surface of a first shaft portion and curved wells or notches on the inner surface of a second shaft portion; and
elastomeric sealant rings and covered radio-opaque marker rings, together forming annular protrusions, at regular intervals along an outer surface of a first shaft portion and complementarily rounded wells within an inner surface of a second shaft portion.

8. The length-adjustable catheter of claim 1 further comprising a kink-protection sleeve covering a portion of the first, fixed-length portion of the shaft.

9. The length-adjustable catheter of claim 1 wherein the inflatable component is a catheter balloon into which liquids and/or gases, including contrast-agent and saline formulations, air, and other such gases and/or liquids, are transferred, under positive pressure, from the inflation port through the inflation lumen.

10. The length-adjustable catheter of claim 1 further comprising annular radio-opaque marker rings positioned at regular intervals along the outer surface of the shaft.

11. The length-adjustable catheter of claim 1, wherein, during length adjustment, the length-adjustable catheter provides optical, haptic, and/or length-adjustment tactile feedback to a treatment provider.

12. The length-adjustable catheter of claim 1, wherein the shaft is provided reinforced by incorporation of threads on the surfaces of one or more shaft portions and components.

13. A length-adjustable catheter comprising:
a shaft having
a first, proximal end,
a second, distal end,
a single-lumen portion having
a first, fixed-length portion that includes the first, proximal end,
a second fixed-length portion that includes the second, distal end,
a length-varying component portion between the first and second fixed-length portions, and
one or more locking features, and
a dual-lumen portion that includes the second, distal end;
a manifold having
a first, distal end mounted over the first, proximal end of the shaft,
a second, proximal end, and
a diameter greater than a diameter of any shaft portion and component;
a guide-wire port in the dual-lumen portion of the shaft;
a guide-wire lumen that extends, through the dual-lumen portion of the shaft, from the guide-wire port to an opening at the second, distal end of the shaft, a linear length axis of the guide-wire lumen parallel to a length axis of the shaft;
an inflation port that extends from the manifold; and
an inflation lumen that extends, through the shaft, from the inflation port to an inflatable component, a length axis of a linear portion of the inflation lumen within the shaft parallel to the length axis of the shaft.

14. The length-adjustable catheter of claim 13 wherein the length-varying component is one of:
a longitudinally and plastically deformable single-lumen shaft portion, having a folding pattern embedded into, or imprinted or molded on, the outer surface to facilitate longitudinal extension and contraction;
one or more fluid-impermeable seals that allow the first, fixed-length portion to slide relative to the second, fixed-length portion while maintaining fluid-impermeability of both the inflation lumen and the guide-wire lumen; and
a third fixed length portion and two or more fluid-impermeable seals that allow the first, fixed-length portion and the second, fixed-length portion to slide relative to the third fixed length portion while maintaining fluid-impermeability of the inflation lumen.

15. The length-adjustable catheter of claim 14 wherein the one or more fluid-impermeable seals each comprises:
an inner sealing annulus on the outer surface of a first shaft portion; and
a complementary outer sealing ring with complementary ramp-like surfaces on the inner surface of a second shaft portion.

16. The length-adjustable catheter of claim 14 wherein the one or more fluid-impermeable seals each comprises:
an annular elastomeric membrane affixed to the outer surface of a first shaft portion that expands against an inner surface a second shaft portion.

17. The length-adjustable catheter of claim 14 wherein shaft portions that slide relative to one another have different diameters.

18. The length-adjustable catheter of claim 13 wherein the inflation lumen and guide-wire lumen are separate lumens, with no fluid communication between the inflation lumen and guide-wire lumen.

19. The length-adjustable catheter of claim 13 wherein each of the one or more locking features comprises one of:
annular, ramp-like features on the outer external surface of a first shaft portion and complementary ramp-like features inscribed within an inner surface of a second shaft portion;
sawtooth-like features circularly disposed around the outer surface of a first shaft portion and complementarily shaped grooves inscribed within an inner surface of a second shaft portion;
circular, rib-like features circularly disposed around the outer surface of a first shaft portion and complementarily circular, rounded, well-like features inscribed within an inner surface of a second shaft portion;
wedge-shaped annular features disposed around the outer surface of a first shaft portion and complementarily V-shaped annular depressions within an inner surface of a second shaft portion;
helical thread-like features circularly on the outer surface of a first shaft portion and complementarily helical depressions or wells within an inner surface of a second shaft portion; and wedge-shaped pins on the outer surface of a first shaft portion and curved wells or notches on the inner surface of a second shaft portion; and
elastomeric sealant rings and covered radio-opaque marker rings, together forming annular protrusions, at regular intervals along an outer surface of a first shaft portion and complementarily rounded wells within an inner surface of a second shaft portion.

20. The length-adjustable catheter of claim 13 further comprising a kink-protection sleeve covering a portion of the first, fixed-length portion of the shaft.

21. The length-adjustable catheter of claim 13 wherein the inflatable component is a catheter balloon into which liquids and/or gases, including contrast-agent and saline formulations, air, and other such gases and/or liquids, are transferred, under positive pressure, from the inflation port through the inflation lumen.

22. The length-adjustable catheter of claim 13 further comprising annular radio-opaque marker rings positioned at regular intervals along the outer surface of the shaft.

23. The length-adjustable catheter of claim 12, wherein, during length adjustment, the length-adjustable catheter provides optical, haptic, and/or length-adjustment tactile feedback to a treatment provider.

24. The length-adjustable catheter of claim 13, wherein the shaft is provided reinforced by incorporation of threads on the surfaces of one or more shaft portions and components.
